(19) United States
(12) Patent Application Publication
(10) Pub. No.: US 2004/0153126 A1
(43) Pub. Date: Aug. 5, 2004

(54) Method and apparatus for treating uterine myoma

(76) Inventor: Takashi Okai, Tokyo (JP)

Correspondence Address: Stanley P Fisher, Reed Smith, Suite 1400, 3110 Fairview Park Drive, Falls Church, VA, 22042-4503, US

(21) Appl. No.: 10/478,873

(22) PCT Filed: Jun. 7, 2001
(86) PCT No.: PCT/JP01/04812

(51) Int. Cl. A61N 1/00

(52) U.S. Cl. 607/1

(57) ABSTRACT

To perform a substantially noninvasive treatment by appropriately occluding a feeding artery of uterine myoma, the conditions of the feeding artery of uterine myoma are grasped by using ultrasonic three-dimensional visual display techniques and the irradiation sites of high intensity focused ultrasound (HIFU) are indicated on the display to thereby occlude the feeding artery which feeds nutrition to the uterine myoma.

### BACKGROUND OF THE INVENTION

The present invention relates to a method and an apparatus for treating uterine myoma.

As medical technologies progress, increased demands have been made on minimally invasive testaments orienting to the quality of life of subjects. Uterine myoma is a disease with a very high incidence and is believed to exist in 20% to 40% of total mature women. There are many subjects with uterine myoma requiring surgery, and operations of uterine myoma hold the majority of abdominal operations in gynecology. However, surgical treatments may invite complications and/or aftereffects at some frequency, and such troubles in surgical treatments often lead to lawsuits. Women who are working in their mature stages increase, and they have strong inhibitions to admission and surgery. As a possible treatment other than the surgery, a hormonal therapy of reducing the level of estrogen, a kind of hormones, in the blood to thereby reduce uterine myoma in size has been developed. However, this treatment cannot be applied for a long time due to its adverse reactions and is employed only as an adjunctive therapy prior to surgery. Ravina attained success of reducing myoma in size by inserting a catheter from the femoral artery into a uterine artery to thereby occlude the uterine artery (Lancet, 346 (1995)). Replications and improvements on this technique have been made in various countries, and its clinical application is being launched in Japan.

Separately, ultrasound exhibits higher penetrance to the depths of a body and a higher energy convergence and affects the whole of a human body less than electromagnetic waves such as laser radiation. Accordingly, ultrasound has been used not only in diagnoses but also in treatments in recent years. Ultrasound application is capable of focusing energy to a minute area on the order of millimeters and inducing local tissue degeneration instantly by its heating action. This technique is clinically applied to the treatment of prostatic hyperplasia (Bihrlre R, J Urol 152, 1994).

### SUMMARY OF THE INVENTION

However, though it is minimally invasive, the technique of occluding a uterine artery using an arterial catheter is a minor operation, in which the inguinal region is incised and the arterial catheter is inserted thereinto, and thereby cannot avoid invasion to some extent and risks accompanied with the invasion. Feeding artery of uterine myoma cannot be selectively occluded according to this technique. In addition, a technique of directly degenerating the uterine myoma tissue by application of high intensity focused ultrasound (HIFU) cannot be significantly applied to the treatment of the uterine myoma. This is because uterine myoma to be treated has a size several times or more larger than that of the prostate gland.

Although causes of uterine myoma have not been clarified, the uterine myoma once occurred is fed with nutrition from a feeding artery and grows, regardless of its cause. If the feeding artery can be appropriately occluded, the size of the uterine myoma can be reduced to thereby mitigate a burden on a subject, as Ravina attained success of reducing myoma in size by inserting a catheter from the femoral artery into a uterine artery to thereby occlude the uterine artery (Lancet, 346 (1995)). Accordingly, demands have been made on a treatment which can mitigate a burden on a subject not by directly degenerating the uterine myoma tissue but by occluding an appropriate point of the feeding artery which feeds nutrition to the uterine myoma with the use of the high intensity focused ultrasound (HIFU).

For example, the fact that small arteries can be occluded by using the high intensity focused ultrasound (HIFU) has been verified in test using animals as described by Rivens (I. M. Euro, J. Ultr, 9, 1999). The conditions of a feeding artery of uterine myoma can be grasped by ultrasonic three-dimensional display technologies which have progressed in recent years. Focussing attention on these points, the present invention makes a proposal of occluding a feeding artery which feed nutrition to uterine myoma by specifying appropriate one or more points of the feeding artery using ultrasound, intensively focussing ultrasonic output to the one or more points and in the vicinity thereof to thereby degenerate the tissue of feeding artery.

### BRIEF DESCRIPTION OF THE DRAWINGS

FIG. 1 is a schematic diagram showing an embodiment of the present invention.

FIG. 2 is a block diagram showing a configuration of ultrasonic irradiation apparatus according to an embodiment of the present invention.

FIGS. 3(A) and 3(B) are each a plan view and a partial sectional view, respectively, showing a configuration of the ultrasonic transducer shown in FIG. 2.

FIGS. 4(A) and 4(B) are each a plan view and a partial sectional view, respectively, showing a configuration of a probe of the ultrasonic irradiation apparatus according to another embodiment.

### DESCRIPTION OF THE PREFERRED EMBODIMENT

Diagnoses, in which ultrasound is applied into the body of a subject, reflected ultrasound is received to produce an image display, have been widely employed. Recently, attempts have been made to apply intense ultrasound to an affected area specified based on the image display to thereby treat a disease. However, it is important that an area to be irradiated with ultrasound should be fee from gas in order to apply ultrasound into the body of a subject effectively. Reviewing this point on the treatment of uterine myoma as in the present invention, the ultrasound cannot be effectively applied into the body in early stages of the uterine myoma in which the subject has no subjective symptoms, since the intestine containing gas exist between the uterine and the abdominal wall.

In contrast, in stages when the uterine myoma progresses and the subject has subjective symptoms, the intestine between the uterine and the abdominal wall is pushed away by the uterine myoma, and thus the uterine myoma can be clearly seen as an image by the application of ultrasound.

The present invention focuses attention on this point and employs the following configuration. Ultrasound is initially applied to uterine myoma of a subject to thereby grasp the conditions of a feeding artery of uterine myoma and to specify an occlusion point of the feeding artery. Next, high intensity focused ultrasound (HIFU) is applied to the specified occlusion point in the feeding artery of uterine myoma to occlude the feeding artery to thereby treat the uterine myoma. In this procedure, the feeding artery of uterine myoma is identified and occluded by the application of the high intensity focused ultrasound (HIFU) using one transducer. Thus, the feeding artery is appropriately occluded.

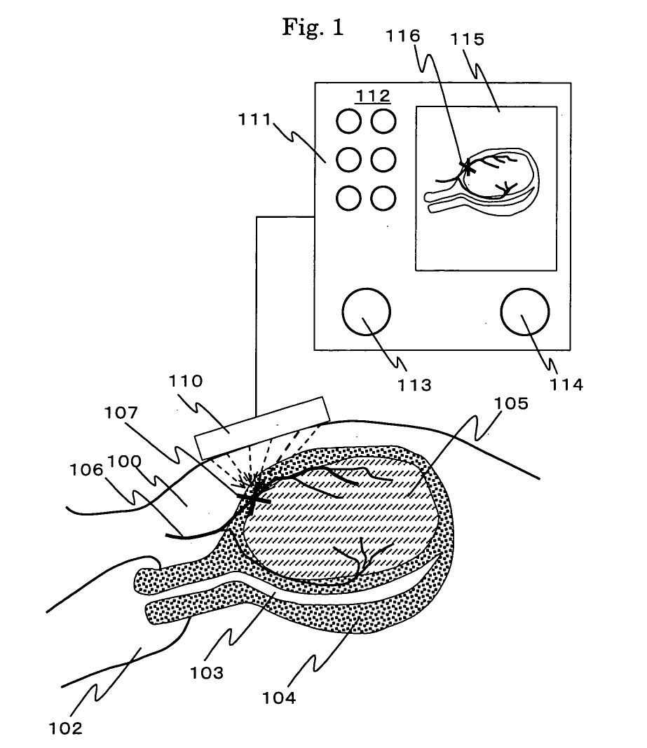

FIG. 1 is a schematic diagram showing an embodiment of the present invention, which illustrates an abdominal wall 100 of a subject to be treated according to the present invention, a vagina 102, a uterine lumen 103, a normal myometrium 104, and uterine myoma 105. A feeding artery 106 is an artery feeding nutrition to the normal myometrium 104 and the uterine myoma 105. A transducer 110 is placed on the abdominal wall 100 of the subject, applies ultrasound to the uterus of the subject, and receives ultrasound reflected from the body of the subject. A control unit 111 is connected to the transducer 110 and controls the ultrasonic application for specifying the occlusion point of the feeding artery of uterine myoma, the receiving of ultrasound reflected from the body, and the irradiation of the high intensity focused ultrasound (HIFU) for occluding the feeding artery of uterine myoma. The control unit 111 includes manual operation buttons 112 for setting conditions and parameters, a monitor screen 115, a trackball 113 for moving a cursor 116 on the monitor screen 115 to any position on the monitor screen 115, and an irradiation button 114 for directing the irradiation of the high intensity focused ultrasound (HIFU) for occluding the feeding artery. The transducer 110 herein is controlled so as to serve as an irradiation probe having a variable focusing point and a variable output. Specific examples of the transducer will be described later.

A doctor who treats uterine myoma places the transducer 110 on the external surface of the abdominal wall 100 of the subject and manipulates the control unit 111 to thereby apply ultrasound to the uterus of the subject. The applied ultrasound is reflected by the uterus, and the transducer 110 receives the reflected ultrasound. The received reflected ultrasound is captured into the control unit 111 and is indicated as an image of the uterus of the subject on the monitor screen 115. In this procedure, the intensity and depth of focus of the ultrasound to be applied to the subject, the brightness and contrast of the display image on the monitor screen 115, and other conditions can be optionally controlled by the manual operation buttons 112.

The doctor observes an image of the uterus and the vicinity thereof displayed on the monitor screen 115 and determines an occlusion point of the feeding artery which is suspected to be effective to reduce the uterine myoma. The cursor 116 can move to any position on the monitor screen 115. The doctor manipulates the trackball 113 and thereby registers the cursor 116 at the determined occlusion point of the feeding artery which is suspected to be effective to reduce the uterine myoma. While holding the cursor 116 at the registered position defined as the occlusion point of the feeding artery, the doctor controls the irradiation intensity of the high intensity focused ultrasound (HIFU) using the manual operation buttons 112 and manipulates the irradiation button 114 to thereby apply the high intensity focused ultrasound (HIFU). Upon the irradiation of the high intensity focused ultrasound (HIFU) by the manipulation of the irradiation button 114, the high intensity focused ultrasound (HIFU) is applied through the abdominal wall 100 to a point of the feeding artery of uterine myoma and to the vicinity thereof corresponding to the registered cursor 116.

According to the present invention, the feeding artery can be occluded by the following three techniques, and the irradiation intensity of the high intensity focused ultrasound (HIFU) is set according to the selected technique. 1) A technique in which heat generated at an irradiation site of the high intensity focused ultrasound acts to degenerate the tissue of vascular wall to constrict a vascular cavity to thereby occlude the same.

2) A technique in which the high intensity focused ultrasound induces cavitation in the vessel, thus free radicals form and damage the vascular endothelium at a position to which the high intensity focused ultrasound has been applied. The damage of the vascular endothelium induces a local arteriosclerosis lesion, which leads to the formation of thrombi and the occlusion of the vessel. According to this technique, the change gradually occurs, and the treatment proceeds gradually, and post-operation care is more important.

3) A technique employing the above two techniques in combination.

Administration, by intravenous injection, of an ultrasonic contrast medium (sensitizer) to the subject is effective to produce more clear images when the doctor observes the image of the uterus and the vicinity thereof displayed on the monitor screen 115 and determines an occlusion point of the feeding artery which is suspected to be effective to reduce the uterine myoma. In this case, microbubbles contained in the ultrasonic contrast medium (sensitizer) play a role, for example, to amplify the cavitation, thereby increase the advantages of the techniques 1) and 2) and is also useful to improve the therapeutic effect.

An example of ultrasonic irradiation apparatus having ultrasonic image pickup means for monitoring an irradiated area is an appropriately modified model of an apparatus disclosed in Japanese Examined Patent Application Publication No. 06-59289. The configuration and operation of the apparatus of this type will be briefly illustrated below.

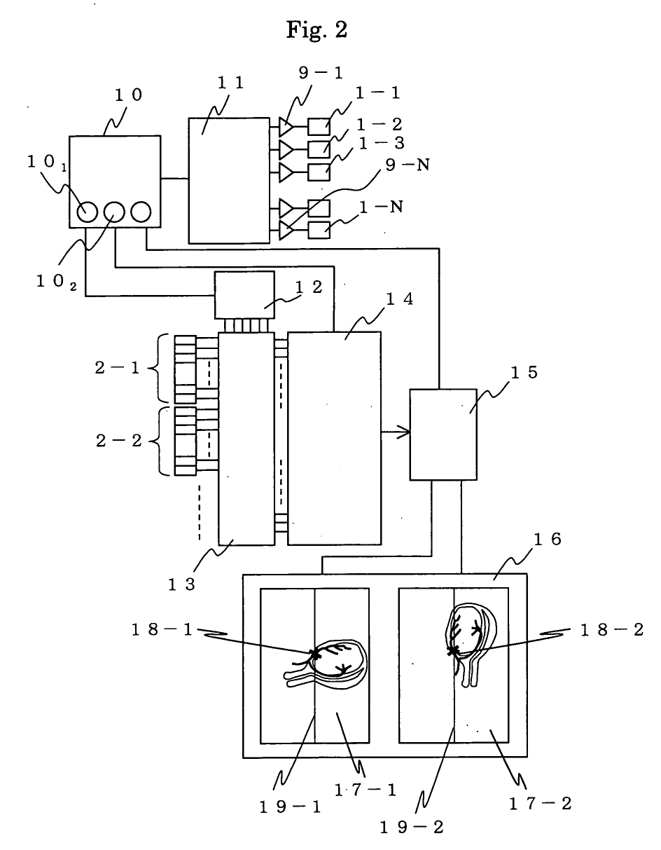

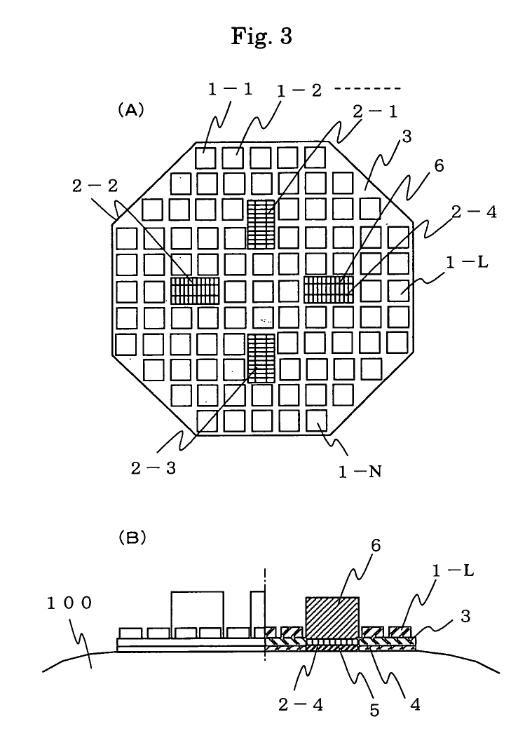

FIG. 2 is a block diagram showing a configuration of an embodiment of the ultrasonic irradiation apparatus, and FIGS. 3(A) and 3(B) are each a plan view and a partial sectional view of a configuration of an ultrasonic transducer constituting the apparatus according to the embodiment.

Initially, the configuration of the ultrasonic transducer will be briefly illustrated with reference to FIG. 3. Irradiation probes 1-1, 1-2, . . . , 1-L, . . . , and 1-N are arranged in an array. Pickup transducers 2-1, 2-2, . . . , and 2-4 each comprise 3 by m pickup probes arranged in a two-dimensional array. A light alloy substrate 3 bears the irradiation probes 1-1, 1-2, . . . , 1-N on its entire surface. A polymeric matching layer 4 is arranged by adhesion on the other side of the light alloy substrate 3. The light alloy substrate 3 is a substrate made of a light alloy and serves as an acoustic matching layer, a heatsink, and a grounding electrode. The combination as a matching layer of the light alloy substrate 3 and the polymeric acoustic matching layer 4 is most effective for a subject to be treated having an acoustic impedance near to that of water. The heatsink is effective to prevent-elevated temperatures due to heat from the irradiation probes upon the application of the ultrasound. The pickup transducers 2-1, 2-2, . . . , and 2-4 are arranged on a pickup probe matching layer 5 as a result of cutting the light alloy substrate 3. In the present embodiment, the pickup transducers 2-1, 2-2, . . . , and 2-4 are symmetrically arranged on the crossing center lines of the light alloy substrate 3. A case 6 houses the pickup probes. The irradiation transducers each have an array structure of probes with a frequency of 500 kHz, the pickup transducers each have an array structure of probes with a center frequency of 3 MHz, and the both are combined.

The overall structure of the ultrasonic irradiation apparatus will be illustrated with reference to the block diagram shown in FIG. 2. A main control circuit 10 corresponds to the control unit 111 in FIG. 1 and generically controls an irradiation transmission control circuit 11 and a pickup transmission control circuit 12 described below. The irradiation transmission control circuit 11 performs arithmetic computations on the phases of alternating current signals for driving the irradiation probes 1-1, 1-2, . . . , and 1-N according to a target position of irradiation designated by the main control circuit 10. Probe element driving circuits 9-1, . . . , and 9-N generate driving signals computed by the irradiation transmission control circuit 11. The irradiation target position is designated by cursors 18-1 and 18-2 on a display screen, details of which will be described later. A display control circuit 15 generates signals for displaying an image of the uterus to be treated based on the ultrasonic signals which have been received by the pickup transducers 2-1, 2-2, . . . , and 2-4 and have been focused by a receiver focusing circuit 14. A display unit 16 displays the image of the uterus to be treated as two sectional images in plural screens 17-1 and 17-2 according to the output of the display control circuit 15. For examples, an image on the screen 17-1 is derived from signals obtained by the pickup probes 2-1 and 2-3, and another image on the screen 17-2 is derived from signals obtained by the pickup probes 2-2 and 2-4. Lines 19-1 and 19-2 represent a line of intersection between a plane corresponding to the line connecting between the pickup probes 2-1 and 2-3 and a plane corresponding to the line connecting between the pickup probes 2-2 and 2-4. Specifically, the images on the two screens intersect with each other at the lines 19-1 and 19-2. The two images are combined to thereby constitute a three-dimensional display. In the figure, a view of sectional structure is difficult to show, and the direction of the view is changed.

The pickup transmission control circuit 12 controls the transmission of the pickup ultrasound. In a mode for pulse echo images, the transmission control circuit 12 generates transmission pulses with respective timing. A transducer amplifier 13 transmits the transmission pulses generated by the transmission control circuit 12 to individual probe elements of the pickup transducers 2-1, 2-2, . . . , and 2-4 to thereby drive these elements. Echo signals are formed due to discontinuous acoustic impedance in a subject to be irradiated according to the transmission pulses, are received by the pickup probes 2-1, 2-2, . . . , and 2-4 and are amplified by the transducer amplifier 13. The receiver focussing circuit 14 focuses the echo signals amplified by the transducer amplifier 13 to thereby process the signals in terms of the position of their occurrence and the intensity of the ultrasound. The output of the receiver focussing circuit 14 is displayed on the display screens of the display unit 16 through the display circuit 15. The receiver focussing circuit 14 has a band-pass filter (not shown), whose center frequency matches with the frequency of the pickup ultrasound. By setting the frequency of the pickup ultrasound 2 times or more the frequency of the irradiation ultrasound, the ultrasound can be captured even during the application of the ultrasound without interference and thereby the irradiation of the ultrasound can be monitored during irradiation.

The irradiation target position is indicated by the cursor 116 in FIG. 1 and is indicated by the cursors 18-1 and 18-2 in FIG. 2. The irradiation target position is decided by manipulating the trackball 113 to thereby control the cursor 116 in FIG. 1, and is decided by controlling the cursor 18-1 on the screen 17-1 in FIG. 2. The main control circuit 10 includes manipulation means 10₁ corresponding to the trackball 113 in FIG. 1, and the irradiation target position is decided by controlling the manipulation means 101 to thereby manipulate the cursor 18-1. The manipulation means 10₁ may be arranged in the display circuit 15. When the display circuit 15 controls the cursor position, a coordinate signal representing the cursor position is transferred from the display circuit 15 to the main control circuit 10. When the cursor 18-1 is controlled on the screen 17-1, the cursor 18-2 on the screen 17-2 moves responding to the movement of the cursor 18-1. When the cursor 18-2 is controlled on the screen 17-2, the cursor 18-1 on the screen 17-1 moves responding to the movement of the cursor 18-2. Which cursor is to be controlled is decided by the doctor by manipulating a selection switch 102. The operation of moving one cursor on one screen while watching the one screen and of allowing the other cursor on the other screen to follow this movement can be easily performed by detecting the X-Y address of the former cursor on the former screen and controlling the X-Y address of the latter cursor on the latter screen to match with the former X-Y address.

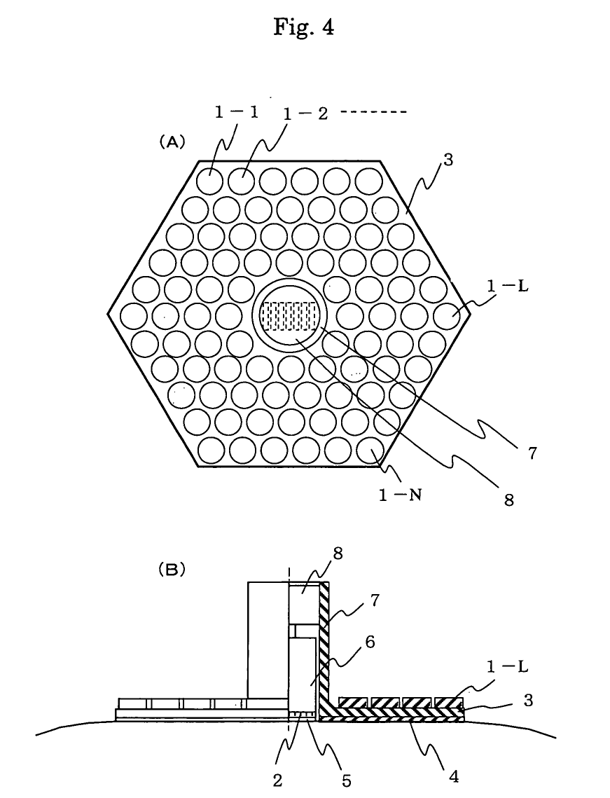

FIGS. 4(A) and 4(B) are each a plan view and a partial sectional view, respectively, showing a configuration of a probe of the ultrasonic irradiation apparatus according to another embodiment. The probe shown in FIG. 4 is different from the probe shown in FIGS. 3(A) and 3(B) in that a pickup linear array probe 2 is mounted via a rotary mechanism 8 to a center part of an irradiation probe. In the present embodiment, a cylindrical support 7 is arranged at the center part of the light alloy substrate 3. On the support 7, the rotary mechanism 8 is to be arranged. The rotary mechanism 8 rotates and controls the pickup probe 2, and the main control circuit 10 is so configured as to control the rotation of pickup probe 2 and to process signals according to the rotation. Different from those illustrated in the first embodiment, the screens 17-1 and 17-2 in the second embodiment are so configured, for example, that the screen 17-1 is a display screen directly corresponding to the output of the pickup probe 2, and the screen 17-2 is a pickup screen and produces a display of an image which is held by the display circuit 15 and has a phase 900 shifted from that in the screen 17-1. According to the present embodiment, the pickup probe 2 can have a smaller area and a smaller number of elements, and the pickup unit can be available at low cost, and the irradiation probe can have an effective area as large as possible. In contrast, the control and signal processing become complicated, but this problem can be easily solved by the application of “a microprocessor”. The description on the entire block including the control system will be omitted. Where necessary, refer to the above-cited publications.

As is described above, according to the present embodiment, the irradiated ultrasound is electronically focused. The apparatus can substantially continuously shift the focus and can obtain focus on a multiplicity of foci concurrently. The embodiments in FIGS. 2 to 4 are illustrated by taking, as an example, an apparatus performing three-dimensional scanning of an irradiation target by electronic scanning using a two-dimensional array as an irradiation ultrasonic probe. However, the present invention can also be applied to a concentric array or a combination use of a fixed-focus probe and a mechanical scanning mechanism. In the above embodiments, a band-pass filter is used in a pickup receiver focussing circuit to avoid interference between the irradiation ultrasound and pickup ultrasound. However, a notch filter for removing a narrow frequency band of the irradiation ultrasound alone can also be used.

Although not illustrated in the above embodiments, the blood flows through the feeding artery, and the feeding artery thereby reflects ultrasound in a manner different from the other still areas. By utilizing this, the feeding artery can be displayed in a color different from the other areas. The specific display of the feeding artery can assist the determination of the occlusion point. In the embodiments shown in the figures, display images in the two display screens are combined to thereby constitute a three-dimensional display. However, recently advanced display technologies can produce an easy-to-see three-dimensional display from one two-dimensional display and can rotate the three-dimensional display image with any axis at the center. By using such a display technique, the ultrasound can be applied more easily.

Among operations of uterine myoma which are believed to be several ten thousand cases a year in Japan, the noninvasive treatment according to the present invention can be possibly applied to many cases. Thus, the invention can contribute to the safety of subjects, can reduce medical expenses necessary for the operations and makes a significant contribution to society.

1. A method for treating uterine myoma, comprising the steps of laying a transducer capable of transmitting and receiving ultrasound to an exterior surface of the abdominal wall of a subject; providing a display of the uterine myoma and a feeding artery of uterine myoma of the subject on a display screen by transmission and reception of ultrasound; designating an occlusion point of the feeding artery of uterine myoma on the display screen; allowing the transducer to apply focused ultrasound to the occlusion point of the feeding artery and to the vicinity thereof; occluding the feeding artery at the occlusion point and in the vicinity thereof acutely or chronically to thereby degenerate and reduce the uterine myoma.

2. The method for treating uterine myoma according to claim 1, further comprising providing a display of the feeding artery in a color different from the other areas based on variations in ultrasonic signal caused by the blood flow in the feeding artery.

3. An apparatus for treating uterine myoma, comprising a transducer capable of being laid to an exterior surface of the abdominal wall of a subject, and transmitting and receiving ultrasound; a display unit for providing a display of uterine myoma and a feeding artery of uterine myoma of the subject on a display screen by transmission and reception of the ultrasound; means for designating an occlusion point of the feeding artery of uterine myoma on the display screen of the display unit; means for degenerating the tissue of the feeding artery by allowing the transducer to apply focused ultrasound to the designated occlusion point of the feeding artery to occlude the feeding artery at the occlusion point and in the vicinity thereof acutely or chronically to thereby degenerate and reduce the uterine myoma; and a control unit for controlling the transmission and receipt of the ultrasound by the transducer, the designation of the occlusion point of the feeding artery on the display screen, and the focusing of the ultrasound.

4. The apparatus for treating uterine myoma according to claim 3, wherein the apparatus is so configured as to provide a display of the feeding artery in a color different from other areas based on variations in ultrasonic signal caused by the blood flow in the feeding artery.
